(19) United States
(12) Patent Application Publication
(10) Pub. No.: US 2012/0237565 A1
(43) Pub. Date: Sep. 20, 2012

(54) PEGYLATED POLYPLEXES CONTAINING TWO OR MORE DIFFERENT POLYMERS FOR POLYNUCLEOTIDE DELIVERY

(75) Inventors: Janni Mirosevich, Tampa, FL (US); Kevin Sill, Tampa, FL (US)

(73) Assignee: INTEZYNE TECHNOLOGIES, INCORPORATED, Tampa, FL (US)

(21) Appl. No.: 13/415,923

(22) Filed: Mar. 9, 2012

Related U.S. Application Data
(60) Provisional application No. 61/452,625, filed on Mar. 14, 2011.

(51) Int. Cl. A61K 31/7088; A61P 29/00; A61P 9/06; A61P 19/02; A61P 9/10; A61P 31/04; A61P 31/12; A61P 25/24; A61P 3/10; A61P 25/08; A61P 31/10; A61P 19/06; A61P 9/12; A61P 33/06; A61P 25/06; A61P 35/00; A61P 25/16; A61P 25/28; A61P 1/00; A61P 37/08; A61P 11/06; A61P 17/06; A61P 19/10; A61P 3/04; A61P 25/18; A61P 27/06; A61K 9/00

(52) U.S. Cl. 424/400; 514/44 R

(57) ABSTRACT

The present invention provides polymers, compositions thereof, and polyplexes comprising said polymers. In particular, cationic polymers, pegylated versions thereof, and polynucleotide containing polyplexes comprising such polymers are provided. The invention further provides methods of using said polymers and polyplexes.

### CROSS REFERENCE TO RELATED APPLICATIONS

The present application claims priority to U.S. provisional patent application Ser. No. 61/452,625, filed Mar. 14, 2011, the entirety of which is hereby incorporated by reference herein.

### FIELD OF THE INVENTION

The present invention relates to the field of polymer chemistry and more particularly to the formation of polynucleotide containing polyplexes and uses thereof.

### BACKGROUND OF THE INVENTION

The development of new therapeutic agents has dramatically improved the quality of life and survival rate of patients suffering from a variety of disorders. However, drug delivery innovations are needed to improve the success rate of these treatments. Specifically, delivery systems are still needed which effectively minimize premature excretion and/or metabolism of therapeutic agents and deliver these agents specifically to diseased cells thereby reducing their potentially adverse effects to healthy cells. Rationally-designed, nanoscopic drug carriers, or “nanovectors,” offer a promising approach to achieving these goals due to their inherent ability to overcome many biological barriers.

### BRIEF DESCRIPTION OF THE DRAWINGS

FIGS. 1A-B Depict a comparison of DNA complexation using PEI or Poly(D/L Asp-DET) Polymers

FIG. 2 depicts a microscopic comparison of Polyplex and PEG-Polyplex size and morphology

FIGS. 3A-B depict co-complexation of DNA using linear PEI and Poly(D/L Asp-DET polymers)

### DETAILED DESCRIPTION OF CERTAIN EMBODIMENTS OF THE INVENTION

### 1. General Description

There are several key factors that limit the use of lipoplexes and polyplexes for in vivo gene delivery applications, particularly when systemic delivery is desired. These include instability of these electrostatic assemblies in high salt environments, irreversible protein binding to the complex that can alter their pharmacokinetic profile, and capture by RES due to excess positive charge. The covalent attachment of poly(ethylene glycol) (PEG) to gene carriers has been shown to address many of these limitations by sterically shielding the complex from unwanted cellular and protein interactions as well as imparting the inherent, stealth properties of PEG. MacLachlan and coworkers have demonstrated that PEG-lipid conjugates, used in conjunction with traditional lipids, can dramatically improve the stability and circulation half-life of DNA-loaded lipoplexes (J. Control. Release, 2006, 112, 280). Similarly, Kissel and coworkers have developed PEG-modified PEI polyplexes that showed enhanced circulation lifetimes when compared to unmodified PEI polyplexes (Pharm. Res., 2002, 19, 810).

PEG has also become a standard choice for the hydrophilic, corona-forming segment of block copolymer polyplexes, and numerous studies have confirmed its ability to reduce RES uptake of micellar delivery systems. See Kwon, G.; Suwa, S.; Yokoyama, M.; Okano, T.; Sakurai, Y.; Kataoka, K. J. Cont. Rel. 1994, 29, 17-23; Caliceti, P.; Veronese, F. M. Adv. Drug Del. Rev. 2003, 55, 1261-1277; Ichikawa, K.; Hikita, T.; Maeda, N.; Takeuchi, Y.; Namba, Y.; Oku, N. Bio. Pharm. Bull. 2004, 27, and 443-444. The ability to tailor PEG chain lengths offers numerous advantages in drug carrier design since studies have shown that circulation times and RES uptake are influenced by the length of the PEG block. In general, longer PEG chains lead to longer circulation times and enhanced stealth properties. In a systematic study of PEG-b-poly(lactic-co-glycolic acid) (PLGA) polyplexs with PEG molecular weights ranging from 5,000-20,000 Da, Langer and coworkers demonstrated that polyplexes coated with 20,000 Da PEG chains were the least susceptible to liver uptake. After 5 hours of circulation, less than 30% of the polyplexs had accumulated in the liver. See Gref, R.; Minamitake, Y.; Peracchia, M. T.; Trubetskoy, V.; Torchilin, V.; Langer, R. Science 1994, 263, 1600-1603.

Two other aspects of a gene delivery system must also be considered: the buffering capacity of the polycation and the intracellular release of the polynucleotide from the polymer.

The present invention describes the preparation of a poly-ion complex (PIC, also known as a polyplex) from two or more polycations with suitable buffering capacity and morphology to allow for polynucleotide release. Mixing plasmid DNA with cationic polymers such as Poly(Asp-DET) results in the formation of nanosized polyplexes that are sub 100 nm in size. Furthermore, DNA polyplexes formed using Poly(Asp-DET) polymers are amenable to surface modifications using N-hydroxysuccinimide (NHS) functionalized 12 kDa PEG. NHS ester groups on PEG react with deprotonated primary amines present on Poly(Asp-DET) polymers to produce stable amide bonds. Physicochemical characterization of the resulting PEG-Polyplexes shows uniform sized nanoparticles that are also smaller than 100 nm. The covalent attachment of PEG does not affect DNA condensation and confers additional stability to nanoparticles, allowing for delivery by systemic administration. The preparation and use of Poly(Asp-DET) polymers is described in United States Patent Application Publication No. US 2011-0229528, filed Mar. 14, 2011, the entirety of which is hereby incorporated herein by reference.

Without wishing to be bound to any particular theory, covalent attachment of PEG, by similar NHS chemistry, to polyplexes formed using either Linear 22 kDa poly(ethylene imine) (PEI) or Branched 25 kDa PEI results in either insufficient polyplex PEG coverage (Linear), or alterations in DNA condensation (Branched). PEIs are known to have a buffering profile that is ideal for endosomal escape. Therefore, without wishing to be bound to any particular theory, it is believed that by complexing with a cationic polymer that exhibits an ideal buffering capacity for endosomal escape with a cationic polymer that has the ability to conjugate with a PEG moiety will allow for a polyplex that has optimized buffering capacity and PEG coverage. In one aspect, the DNA is initially complexed with PEI then further complexed with Poly(Asp-DET), followed by the conjugation of PEG to the polyplex to give polyplexes with mamixal buffer capability and sufficient PEG coverage for increased in vivo stability.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “portion” or “block” refers to a repeating polymeric sequence of defined composition. A portion or a block may consist of a single monomer or may be comprise of on or more monomers, resulting in a “mixed block”.

One skilled in the art will recognize that a monomer repeat unit is defined by parentheses depicted around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For Example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the term “polycation” or “cationic polymer” may be used interchangeably and refer to a polymer possessing a plurality of ionic charges. In some embodiments polycation also refers to a polymer that possess a plurality of functional groups that can be protonated to obtain a plurality of ionic charges. For clarity, a polymer that contains a plurality of amine functional groups will be referred to as a polycation or a cationic polymer within this application.

In certain embodiments, a provided cation is suitable for polynucleotide encapsulation. As used herein, the term “polynucleotide” refers to DNA or RNA. In some embodiments, a polynucleotide is a short interfering RNA (siRNA), a microRNA (miRNA), a plasmid DNA (pDNA), a short hairpin RNA (shRNA), messenger RNA (mRNA), antisense RNA (asRNA), to name a few, and encompasses both the nucleotide sequence and any structural embodiments thereof, such as double stranded, single stranded, helical, hairpin, etc.

As used herein, the terms “polynucleotide-loaded” and “encapsulated,” and derivatives thereof, are used interchangeably. In accordance with the present invention, a “polynucleotide-loaded” polyplex refers to a polyplex having one or more polynucleotides situated within the core of the polyplex. This is also referred to as a polynucleotide being “encapsulated” within the polyplex.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit is in the L-configuration. In other embodiments, the amino acid units are a mixture of D and L configurations. Such poly(amino acids) include those having suitably protected functional groups. For Example, amino acid monomers may have hydroxyl or amino moieties that are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In still other embodiments, amino acid blocks of the present invention include random amino acid blocks, i.e., blocks comprising a mixture of amino acid residues.

As used herein, the phrase “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For Example, a suitably protected tyrosine hydroxyl group can render that tyrosine nonpolar and hydrophobic by virtue of protecting the hydroxyl group.

As used herein, the term “D,L-mixed poly(amino acid)” refers to a poly(amino acid) wherein the poly(amino acid) consists of a mixture of amino acids in both the D- and L-configurations. It is well established that homopolymers and copolymers of amino acids, consisting of a single stereoisomer, may exhibit secondary structures such as the α-helix or β-sheet. See α-Aminoacid-N-Caroboxy-Anhydrides and Related Heterocycles, H. R. Kricheldorf, Springer-Verlag, 1987. For Example, poly(L-benzyl glutamate) typically exhibits an α-helical conformation; however this secondary structure can be disrupted by a change of solvent or temperature (see Advances in Protein Chemistry MVI, P. Urnes and P. Doty, Academic Press, New York 1961). The secondary structure can also be disrupted by the incorporation of structurally dissimilar amino acids such as β-sheet forming amino acids (e.g. proline) or through the incorporation of amino acids with dissimilar stereochemistry (e.g. mixture of D and L stereoisomers), which results in poly(amino acids) with a random coil conformation. See Sakai, R.; Ikeda; S.; Isemura, T. Bull Chem. Soc. Japan 1969, 42, 1332-1336, Paolillo, L.; Temussi, P. A.; Bradbury, E. M.; Crane-Robinson, C. Biopolymers 1972, 11, 2043-2052, and Cho, I.; Kim, J. B.; Jung, H. J. Polymer 2003, 44, 5497-5500.

As used herein, the term “tacticity” refers to the stereochemistry of the poly(amino acid). A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer. Polymer tacticity is described in more detail in “Principles of Polymerization”, 3rd Ed., G. Odian, John Wiley & Sons, New York: 1991, the entire contents of which are hereby incorporated by reference.

As used herein, the phrase “unnatural amino acid side-chain group” refers to the side-chain group of amino acids not included in the list of 20 amino acids naturally occurring in proteins, as described above. Such amino acids include the D-isomer of any of the 20 naturally occurring amino acids. Unnatural amino acids also include homoserine, ornithine, norleucine, and thyromine. Other unnatural amino acids side-chains are well known to one of ordinary skill in the art and include unnatural aliphatic side chains. Other unnatural amino acids include modified amino acids, including those that are N-alkylated, cyclized, phosphorylated, acetylated, amidated, azidylated, labelled, and the like. In some embodiments, an unnatural amino acid is a D-isomer. In some embodiments, an unnatural amino acid is a L-isomer.

As used herein, the phrase “amine-containing amino acid side-chain group” refers to natural or unnatural amino acid side-chain groups, as defined above, which comprise an amine moiety. The amine moiety may be primary, secondary, tertiary, or quaternary, and may be part of an optionally substituted group aliphatic or optionally substituted aryl group.

As used herein, the phrase N to P (N/P or N:P) refers to the ratio of protonatable nitrogens (N) to negatively charged phosphate groups in the DNA or RNA backbone (P).

The term “aliphatic” or “aliphatic group,” as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon. This includes any omidized form of nitrogen, sulfur, phosphorus, or silicon; the quaternized form of any basic nitrogen, or; a substitutable nitrogen of a heterocyclic ring including ═N— as in 3,4-dihydro-2H-pyrrolyl, —NH— as in pyrrolidinyl, or ═N(R^{†})— as in N-substituted pyrrolidinyl.

The term “unsaturated,” as used herein, means that a moiety has one or more units of unsaturation.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein at least one ring in the system is aromatic and wherein each ring in the system contains three to seven ring members. The term “aryl” may be used interchangeably with the term “aryl ring.”

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R, -(haloR.), —(CH₂)₀₋₂OH, —(CH₂)₀₋₂OR, —(CH₂)₀₋₂CH(OR.)₂; —O(haloR.), —CN, —N₃, —(CH₂)₀₋₂C(O)R, —(CH₂)₀₋₂C(O)OH, —(CH₂)₀₋₂C(O)OR, —(CH₂)₀₋₂SR, —(CH₂)₀₋₂SH, —(CH₂)₀₋₂NH₂, —(CH₂)₀₋₂NHR, —(CH₂)₀₋₂NR.₂, —NO₂, —SiR.₃, —OSiR.₃, —C(O)SR, —(C₁₋₄ straight or branched alkylene)C(O)OR, or —SSR. wherein each R. is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. A suitable tetravalent substituent that is bound to vicinal substitutable methylene carbons of an “optionally substituted” group is the dicobalt hemacarbonyl cluster represented by

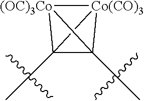

when depicted with the methylenes which bear it.

Suitable substituents on the aliphatic group of R* include halogen, —R, -(haloR.), —OH, —OR, —O(haloR.), —CN, —C(O)OH, —C(O)OR, —NH₂, —NHR, —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R, -(haloR.), —OH, —OR, —O(haloR.), —CN, —C(O)OH, —C(O)OR, —NH₂, —NHR, —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-omopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzylomocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl)acetal, 1,3-diomanes, 1,3-diomolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include omazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Unless otherwise stated, structures depicted herein are also meant to include all isomeric (e.g., enantiomeric, diastereomeric, and geometric (or conformational)) forms of the structure; for Example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For Example, compounds having the present structures except for the replacement of hydrogen by deuterium or tritium, or the replacement of a carbon by a ¹³C- or ¹⁴C-enriched carbon are within the scope of this invention. Such compounds are useful, for Example, in neutron scattering experiments, as analytical tools, or probes in biological assays.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g Fe₃O₄ and Fe₂O₃) particles. Similarly, semiconducting nanoparticles (e.g. cadmium selenide, cadmium sulfide, cadmium telluride) are useful as fluorescent labels. Other metal nanoparticles (e.g colloidal gold) also serve as primary labels.

“Secondary” labels include moieties such as biotin, or protein antigens, that require the presence of a second compound to produce a detectable signal. For Example, in the case of a biotin label, the second compound may include streptavidin-enzyme conjugates. In the case of an antigen label, the second compound may include an antibody-enzyme conjugate. Additionally, certain fluorescent groups can act as secondary labels by transferring energy to another compound or group in a process of nonradiative fluorescent resonance energy transfer (FRET), causing the second compound or group to then generate the signal that is detected.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label,” “fluorescent group,” “fluorescent compound,” “fluorescent dye,” and “fluorophore,” as used herein, refer to compounds or moieties that absorb light energy at a defined excitation wavelength and emit light energy at a different wavelength. Examples of fluorescent compounds include, but are not limited to: Alema Fluor dyes (Alema Fluor 350, Alema Fluor 488, Alema Fluor 532, Alema Fluor 546, Alema Fluor 568, Alema Fluor 594, Alema Fluor 633, Alema Fluor 660 and Alema Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-M-rhodamine (ROM), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Temas Red, Temas Red-M.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate,” as used herein refers to any material or macromolecular complex to which a functionalized end-group of a block copolymer can be attached. Examples of commonly used substrates include, but are not limited to, glass surfaces, silica surfaces, plastic surfaces, metal surfaces, surfaces containing a metallic or chemical coating, membranes (e.g., nylon, polysulfone, silica), micro-beads (eg., latem, polystyrene, or other polymer), porous polymer matrices (e.g., polyacrylamide gel, polysaccharide, polymethacrylate), macromolecular complexes (e.g., protein, polysaccharide).

The term “fusogenic peptide” refers to a peptide sequence that promotes escape from endolysomal compartments. Great efforts have been undertaken to further enhance endolysosomal escape and thus prevent lysosomal degradation. A key strategy has been adapted from viral elements that promote escape from the harsh endolysosomal environment and deliver their genetic information intact into the nucleus. Apart from complete virus capsids and purified capsid proteins, short amino acid sequences derived from the N-terminus of Haemophilus Influenza Haemagglutinin-2 have also been shown to induce pH-sensitive membrane disruption, leading to improved transfection of DNA-polycation polymer complexes in vitro. One such Example is the INF7 peptide (GLFGAIAGFIENGWEGMIDGGGC). At neutral pH (pH 7.0) the INF peptide forms a random coil structure without fusogenic activity. However, this peptide undergoes a conformational change into an amphipathic α-helim at pH 5.0 and aggregates resulting in the formation of pores that destabilize endosomal membranes causing vesicle leakage. Indeed, the INF7 peptide has been used in combination with polymer based delivery systems and shown to tremendously enhance gene transfection activity without affecting cell cytotomicity. Other synthetic fusogenic peptides may be used to aid endosome escape of our polymers, such as GALA (WEAALAEALAEALAEHLAEALAEALEALAA) and KALA (WEAKLAKALAKALAKHLAKALAKALKACEA) peptides. These peptides have been shown to successfully promote extensive membrane destabilization and subsequently, contribute to transfection enhancement.

The term “oligopeptide”, as used herein refers to any peptide of 2-65 amino acid residues in length. In some embodiments, oligopeptides comprise amino acids with natural amino acid side-chain groups. In some embodiments, oligopeptides comprise amino acids with unnatural amino acid side-chain groups. In certain embodiments, oligopeptides are 2-50 amino acid residues in length. In certain embodiments, oligopeptides are 2-40 amino acid residues in length. In some embodiments, oligopeptides are cyclized variations of the linear sequences. In other embodiments, oligopeptides are 3-15 amino acid residues in length.

As used herein, the term “targeting group” refers to any molecule, macromolecule, or biomacromolecule that selectively binds to receptors that are expressed or over-expressed on specific cell types. Targeting groups are well known in the art and include those described in International application publication number WO 2008/134731, published Nov. 6, 2008, the entirety of which is hereby incorporated by reference. In some embodiments, the targeting group is a moiety selected from folate, a Her-2 binding peptide, a urokinase-type plasminogen activator receptor (uPAR) antagonist, a CMCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide.

As used herein, the term “electron withdrawing group” refers to a group characterized by a tendency to attract electrons. Exemplary such groups are known in the art and include, by way of nonlimiting Example, halogen, nitrile, nitro, carbonyl, and conjugated carbonyl.

### 3. Description of Exemplary Embodiments

A. Cationic Polymers

In certain embodiments, one or more of the cationic polymers described above contains a mixture of primary and secondary amine groups on the side chain of the poly(amino acid). One of ordinary skill in the art will recognize that primary amine groups interact with phosphates in the polynucleotide to form the polyplex, whereas secondary amine groups function as a buffering group, or proton sponge, which aids in endosomal escape via endosome disruption. Ideally, one would select the optimum number of primary and secondary amines to both complex the polynucleotide and allow for sufficient endosomal escape, while limiting cytotoxicity.

In certain embodiments, the present invention provides a cationic polymer of formula I, or a salt thereof:

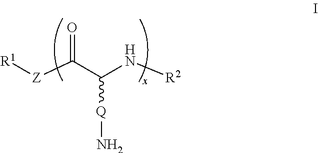

wherein:
m is 10-250;
Each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   each Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
R¹ is hydrogen, —N₃, —CN, a suitable amine protecting group, a protected aldehyde, a protected hydroxyl, a suitable hydroxyl protecting group, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide.

In certain embodiments, the x group is about 10 to about 250. In certain embodiments, the x group is about 25. In other embodiments x is about 10 to about 50. In other embodiments, x is about 50. According to yet another embodiment, x is about 75. In other embodiments, x is about 100. In certain embodiments, x is about 40 to about 80. In other embodiments, x is selected from 10±5, 15±5, 25±5, 50±5, 75±10, 100±10, or 125±10.

In certain embodiments, the Z group is a —NH— group. In certain embodiments, the Z group is a valence bond.

In certain embodiments, the R¹ group is a saturated or unsaturated alkyl chain. In other embodiments, the R¹ group is a pentyl group. In other embodiments, the R¹ group is a hemyl group. In other embodiments, the R¹ group is a hydrogen atom. In other embodiments, the R¹ group is a quaternized triethylamine group.

In certain embodiments, the R² group is an acetyl group. In another embodiment, the R² group is a hydrogen atom.

In certain embodiments, the Q group is a chemical moiety representing an oligomer of ethylene amine, —(NH₂—CH₂—CH₂)—. In other embodiments, the Q group is —(CH₂—CH₂—CH₂)— such that the side chain represents ornithine. In other embodiments, the Q group is —(CH₂—CH₂—CH₂—CH₂)— such that the side chain represents lysine. In certain embodiments, Q is a branched alkylene chain wherein one or more methine carbons is replaced with a nitrogen atom to form a trivalent amine group. Specific examples of Q groups can be found in Table 1a, Table 1b, and Table 1c.

TABLE 1a

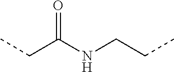

i

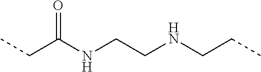

ii

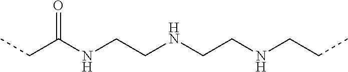

iii

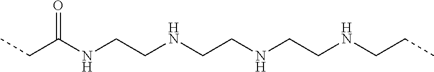

iv

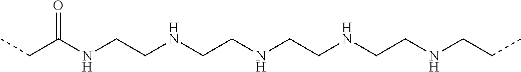

v

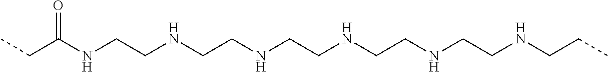

vi

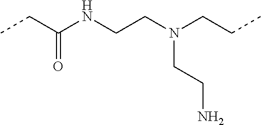

vii

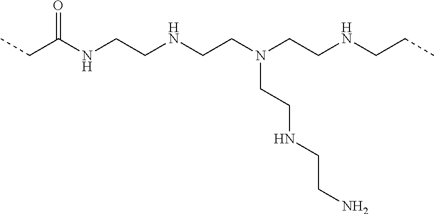

viii

TABLE 1b

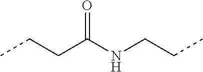

ix

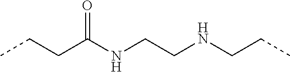

x

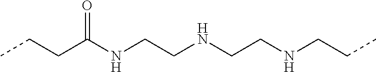

xi

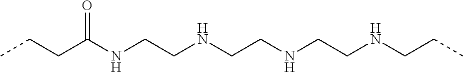

xii

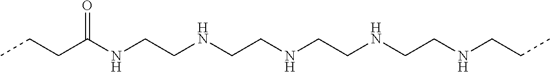

xiii

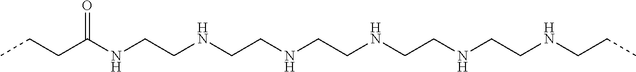

xiv

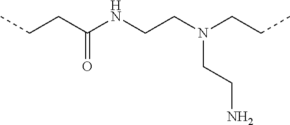

xv

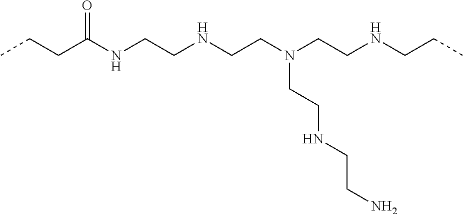

xvi

TABLE 1c

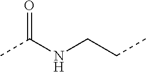

xvii

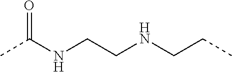

xviii

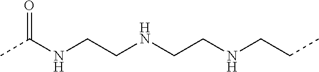

xix

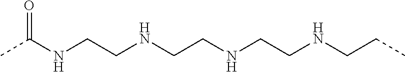

xx

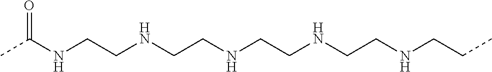

xxi

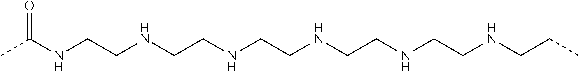

xxii

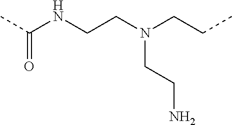

xxiii

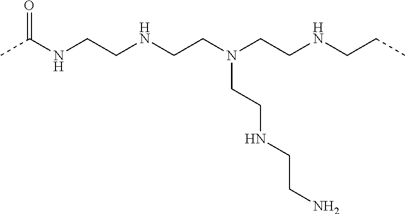

xxiv

One skilled in the art will recognize that the stereochemistry of the poly(amino acid) represented in Formula I is undefined. It is understood that this depiction can represent an all L conformation, an all D conformation, or any random mixture of D and L isomers.

Exemplary polymers, or salts thereof, of Formula I are set forth in Table 2, wherein m is 10-250 and y is 10-250.

TABLE 2

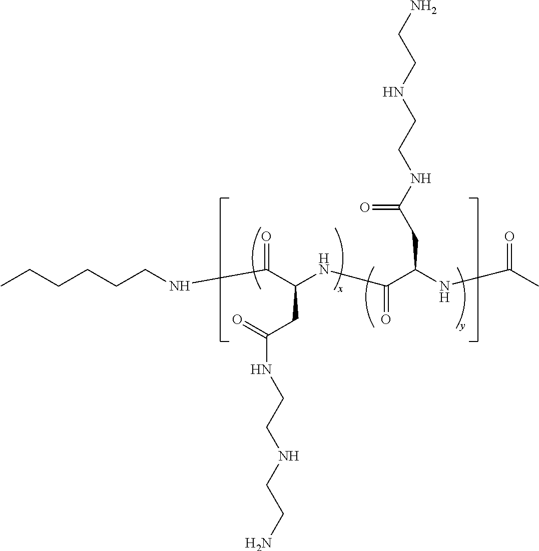

i

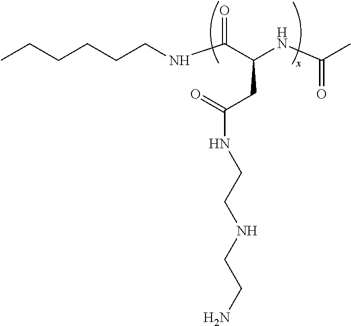

j

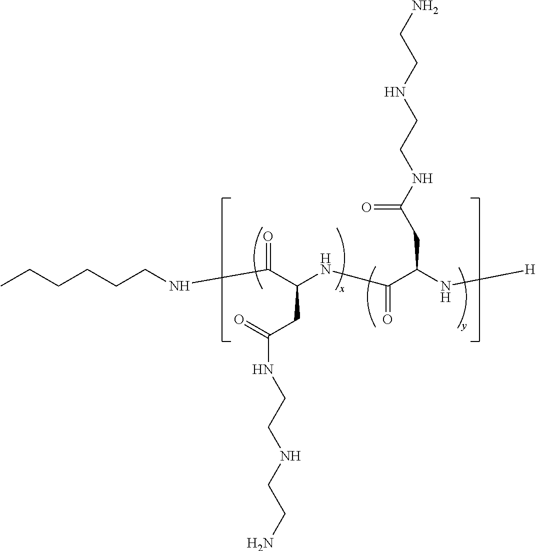

k

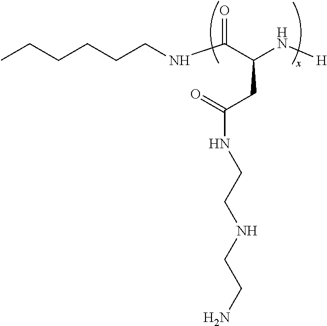

l

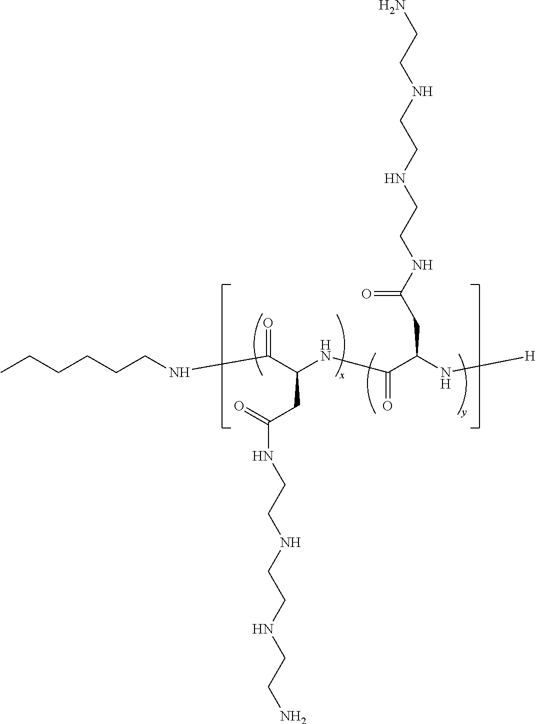

m

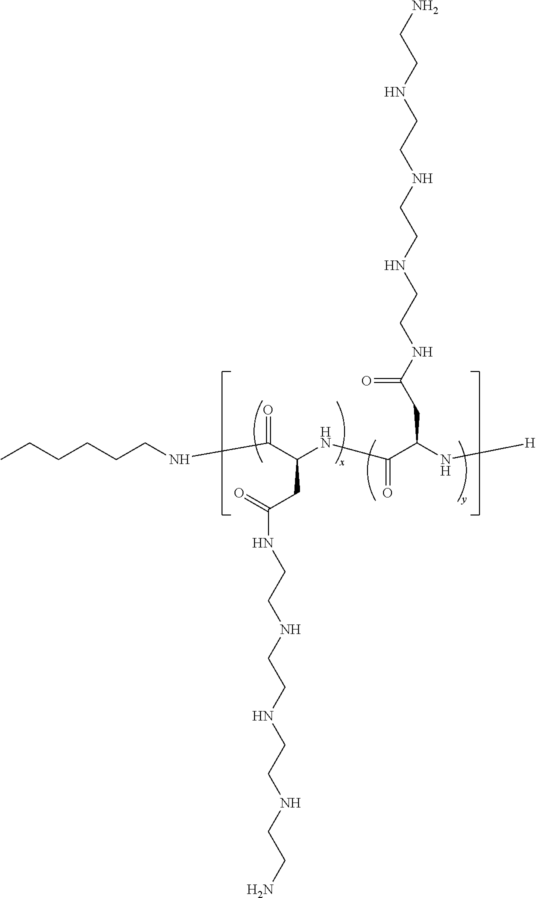

n

In certain embodiments, the present invention provides a cationic polymer of formula II, or a salt thereof:

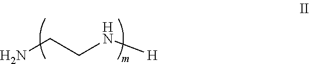

wherein:

m is 10-20,000.

In certain embodiments, the m group is about 10 to about 20,000. In certain embodiments, the m group is about 18. In certain embodiments, the m group is about 27. In certain embodiments, the m group is about 41. In certain embodiments, the m group is about 227. In certain embodiments, the m group is about 500. In certain embodiments, the m group is about 568. In certain embodiments, the m group is about 1363. In certain embodiments, the m group is about 17,045. In other embodiments m is about 10 to about 50. In other embodiments, m is about 200 to about 300. According to yet another embodiment, m is about 450 to about 600. In other embodiments, m is about 1000 to about 1500. In certain embodiments, m is about 15,000 to about 20,000. In other embodiments, m is selected from 18±5, 27±5, 41±5, 227±20, 500±50, 568±50, 1363±100, or 17,045±2,000.

One skilled in the art will recognize that that formula II represents linear poly(ethylene imine) (PEI), also known as poly(iminoethylene), polyaziridine, or poly[(imino-(1,2-ethandiyl)]. Linear PEI is commercially available from Fermetas Life Sciences (Glen Burnie, Md.) as ExGen 500 ™ in vivo Transfection Reagent and from PolyPlus (New York, N.Y.) as JetPEI™

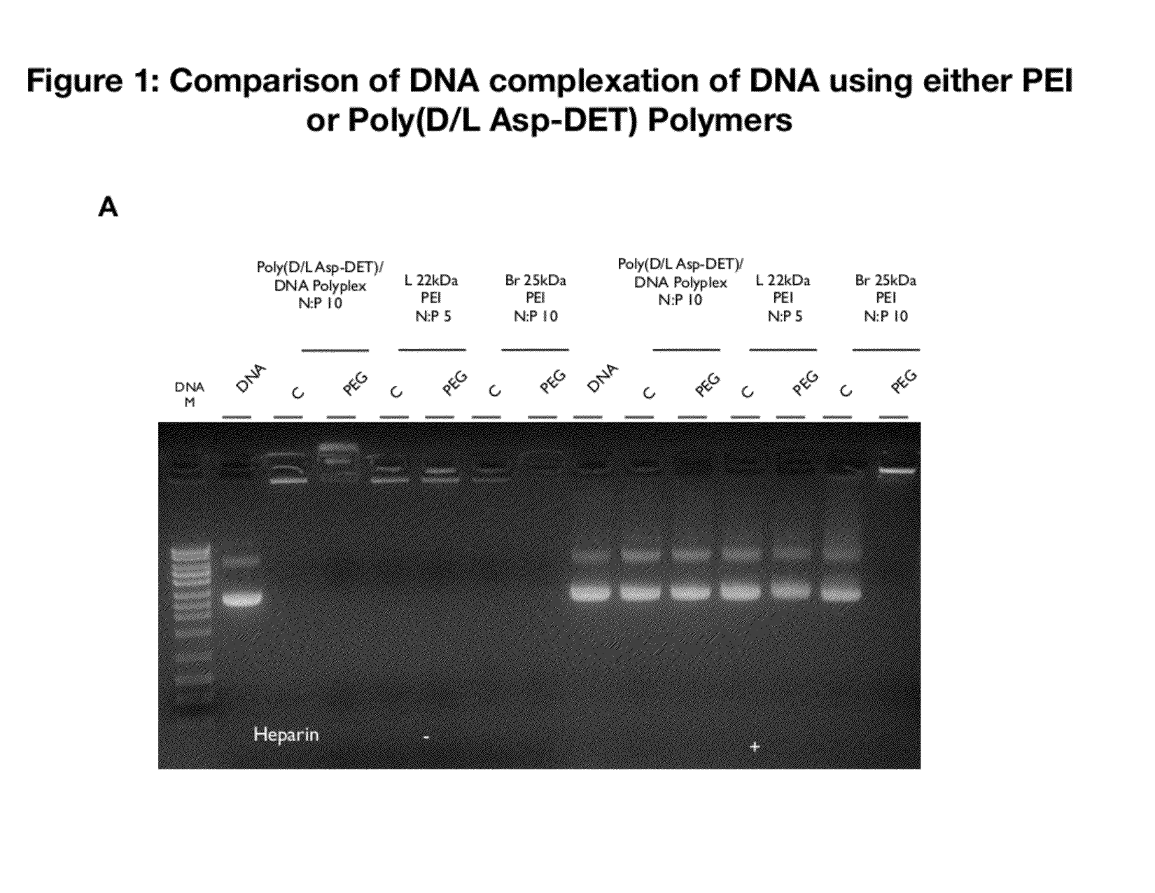

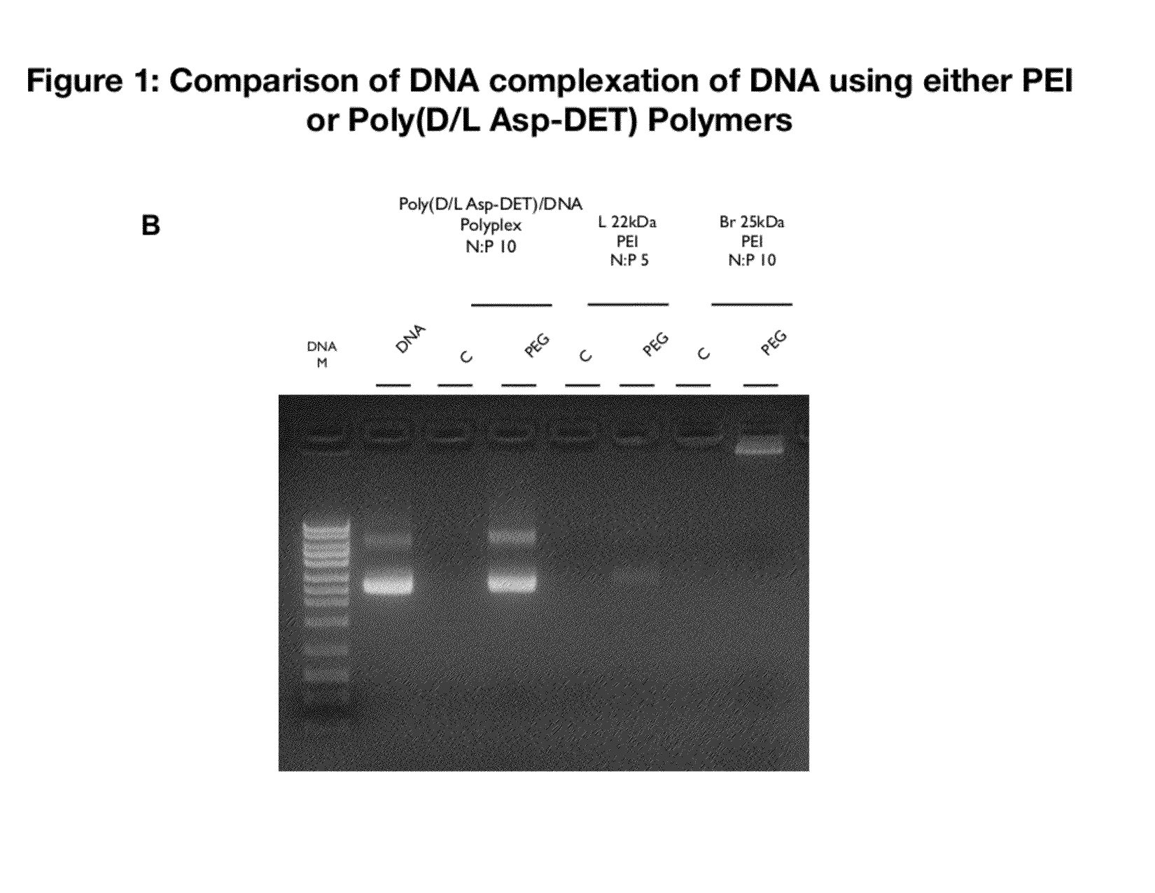

In certain embodiments, the present invention provides a cationic polymer of branched poly(ethylene imine) Branched PEI is well known in the literature (See Kursa, M., G. F. Walker, et al. Bioconjugate Chemistry, 2003, 222-231 and Wightman, R. K. et. al.; The Journal of Gene Medicine, 2001, 3, 362-372) and commercially available. For example, Sigma Aldrich (St. Louis, Mo.) offers molecular weights (number average) of 800, 1,200, 1,800, 25,000 and 60,000 daltons. Branched polyethylene imine is a hyperbranched polymer wherein each amine group can be attached to two protons, representing a terminal amine; to a proton and another ethylene imine group, forming a linear ethylene imine repeat; or to two ethylene imine groups, representing a branch point. Due to its highly branched nature, the exact polymer morphology is very difficult to determine. For clarity, selected branched poly(ethylene imine) structures are shown below in FIG. 1.

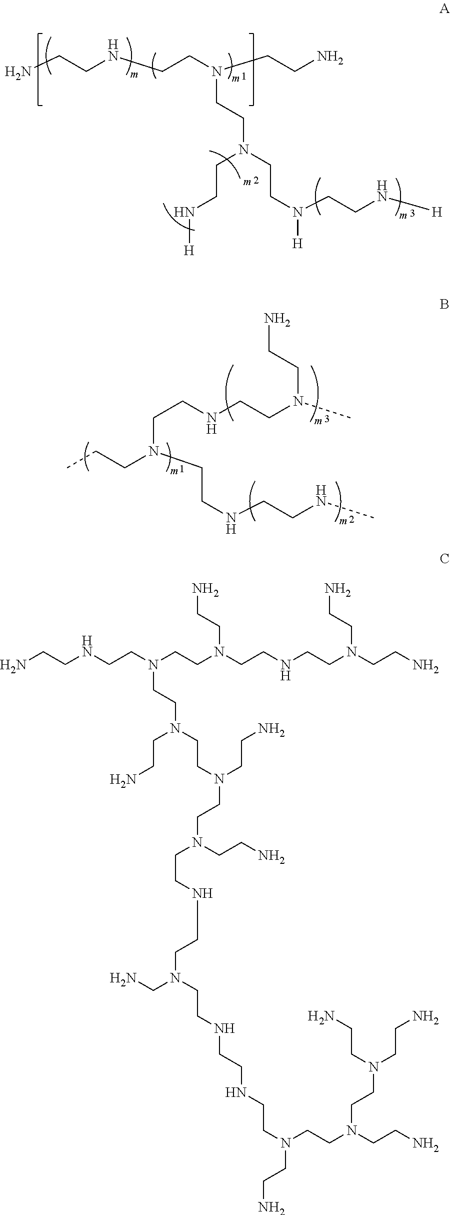

In certain embodiments, the present invention provides a cationic polymer of PEI, linear PEI, branched PEI, poly(arginine), poly(leucine), or poly(ornithine). The molecular weights of each of these polymers may range from 500 to 1,000,000 daltons.

B. Polynucleotide Encapsulation

The present invention provides the preparation of a polyplex formed by the addition of two or more cationic polymers and a polynucleotide.

In water, such cationic copolymers co-assemble with polynucleotides through electrostatic interactions between the cationic side chains of the polymer and the anionic phosphates of the polynucleotide to form a polyplex. In some cases, the number of phosphates on the polynucleotides may exceed the number of cationic charges on the multiblock copolymer. It will be appreciated that multiple polymers may be used to achieve charge neutrality (i.e. N/P=1) between the polymer and encapsulated polynucleotide. It will also be appreciated that when an excess of polymer is used to encapsulate a polynucleotide, the polymer/polynucleotide complex can possess an overall positive charge (i.e. N/P>1).

As described herein, polyplexes of the present invention can be prepared with any polynucleotide agent. In one embodiment, the encapsulated polynucleotide is a plasmid DNA (pDNA). As used herein, pDNA is defined as a circular, double-stranded DNA that contains a DNA sequence (cDNA or complementary DNA) that is to be expressed in cells either in culture or in vivo. The size of pDNA can range from 3 kilo base pairs (kb) to greater than 50 kb. The cDNA that is contained within plasmid DNA is usually between 1-5 kb in length, but may be greater if larger genes are incorporated. pDNA may also incorporate other sequences that allow it to be properly and efficiently expressed in mammalian cells, as well as replicated in bacterial cells. In certain embodiments, the encapsulated pDNA expresses a therapeutic gene in cell culture, animals, or humans that possess a defective or missing gene that is responsible for and/or correlated with disease.

In certain embodiments, an encapsulated polynucleotide is capable of silencing gene expression via RNA interference (RNAi). As defined herein, RNAi is a cellular mechanism that suppresses gene expression during translation and/or hinders the transcription of genes through destruction of messenger RNA (mRNA). Without wishing to be bound by any particular theory, it is believed that endogenous double-stranded RNA located in the cell are processed into 20-25 nt short-interfering RNA (siRNA) by the enzyme Dicer. siRNA subsequently binds to the RISC complex (RNA-induced silencing nuclease complex), and the guide strand of the siRNA anneals to the target mRNA. The nuclease activity of the RISC complex then cleaves the mRNA, which is subsequently degraded (Nat. Rev. Mol. Cell Biol., 2007, 8, 23).

In some embodiments, an encapsulated polynucleotide is a siRNA. As used herein, siRNA is defined as a linear, double-stranded RNA that is 20-25 nucleotides (nt) in length and possesses a 2 nt, 3′ overhang on each end which can induce gene knockdown in cell culture or in vivo via RNAi. In certain embodiments, the encapsulated siRNA suppresses disease-relevant gene expression in cell culture, animals, or humans.

In certain embodiments, the encapsulated polynucleotide is pDNA that expresses a short-hairpin RNA (shRNA). As used herein, shRNA is a linear, double-stranded RNA, possessing a tight hairpin turn, which is synthesized in cells through transfection and expression of a exogenous pDNA. Without wishing to be bound by any particular theory, it is believed that the shRNA hairpin structure is cleaved to produce siRNA, which mediates gene silencing via RNA interference. In certain embodiments, the encapsulated shRNA suppresses gene expression in cell culture, animals, or humans that are responsible for a disease via RNAi.

In certain embodiments, the encapsulated polynucleotide is a microRNA (miRNA). As used herein, miRNA is a linear, single-stranded RNA that ranges between 21-23 nt in length and regulates gene expression via RNAi (Cell, 2004, 116, 281). In certain embodiments, an encapsulated miRNA suppresses gene expression in cell culture, animals, or humans that are responsible for a disease via RNAi.

In another embodiment, an encapsulated polynucleotide is a messenger RNA (mRNA). As used herein, mRNA is defined as a linear, single stranded RNA molecule, which is responsible for translation of genes (from DNA) into proteins. In certain embodiments, the encapsulated mRNA is encoded from a plasmid cDNA to serve as the template for protein translation. In certain embodiments, an encapsulated mRNA translates therapeutic proteins, in vitro and/or in vivo, which can treat disease.

In certain embodiments, an encapsulated polynucleotide is an antisense RNA (asRNA). As used herein, asRNA is a linear, single-stranded RNA that is complementary to a targeted mRNA located in a cell. Without wishing to be bound by any particular theory, it is believed that asRNA inhibits translation of a complementary mRNA by pairing with it and obstructing the cellular translation machinery. It is believed that the mechanism of action for asRNA is different from RNAi because the paired mRNA is not destroyed. In certain embodiments, an encapsulated asRNA suppresses gene expression in cell culture, animals, or humans that are responsible for a disease by binding mRNA and physically obstructing translation.

In certain embodiments, the N/P ratio will be greater than 1. In certain embodiments, the N/P ratio will be range 2 to 50. In some embodiments, the N/P ratio will be selected from 2, 3, or 4. In certain embodiments, the N/P ratio is 5. In yet other embodiments, the N/P ratio is 10. In some embodiments, the N/P ratio is selected from 15, 20, 25, 30, 35, 40, or 50. In other embodiments, the N/P ratio is from 5 to 10. In certain embodiments, the N/P ratio is about 5 or about 10. In yet other embodiments, the N/P ratio is from 4 to 15.

In certain embodiments, the present invention provides a polyplex having a polynucleotide encapsulated therein, comprising a cationic polymer of formula I and PEI.

In certain embodiments, the present invention provides a polyplex having a polynucleotide encapsulated therein, comprising a cationic polymer of formula I and linear PEI.

In certain embodiments, the present invention provides a polyplex having a polynucleotide encapsulated therein, comprising a cationic polymer of formula I and branched PEI.

In certain embodiments, the present invention provides a polyplex having a polynucleotide encapsulated therein, comprising a cationic polymer of formula I and poly(arginine).

In certain embodiments, the present invention provides a polyplex having a polynucleotide encapsulated therein, comprising a cationic polymer of formula I and poly(lysine).

In certain embodiments, the present invention provides a polyplex having a polynucleotide encapsulated therein, comprising a cationic polymer of formula I and poly(ornithine).

C. Polyplex PEGylation

The present invention further provides the preparation of a polyplex formed by the addition of two or more cationic polymers and a polynucleotide, followed by the covalent attachment of PEG to the polyplex to form a PEG-conjugated polyplex.

One of ordinary skill in the art will recognize that multiple avenues exist to conjugate the PEG onto the polyplex. Generally, excess amines present within the polyplex will react with suitable electrophiles to form covalent bonds. Suitable electrophiles include, but are not limited to, maleimides, activated esters, esters, and aldehydes. It is also important to recognize that the pH of the solution will affect the reactivity of the excess amines present within the polyplex. At low pH, the amines will predominately exist as an ammonium salt, and the reaction rate of the ammonium salt with the electrophile will be very low. However, as the pH approaches basic conditions (>7), the amines will have a higher percentage of free amine compared to ammonium salts. When the percentage of free amines increases, the reaction rate with a suitable electrophile will also increase. Thus, it is advantageous to select a pH that allows for the highest reaction rate (basic pH) without causing an adverse effect to the polynucleotide. In some embodiments, the pH of the PEGylation reaction solution is 4.0-9.0. In some embodiments, the pH of the PEGylation reaction solution is 5.0-6.0. In other embodiments, the pH of the PEGylation reaction solution is 6.0-7.0. In some embodiments, the pH of the PEGylation reaction solution is 7.0-8.0. In yet other embodiments, the pH of the PEGylation reaction solution is about 7.0. In another embodiment, the pH of the PEGylation reaction solution is about 7.5. In yet another embodiments, the pH of the PEGylation reaction solution is about 7.4.

In certain embodiments, the present invention provides a cationic polymer of formula IV or a salt thereof:

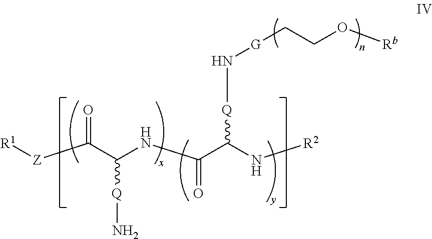

wherein each of R¹, Q, Z, m, and R² is as defined above and as described in classes and subclasses herein, both singly and in combination;
   y is 1-200;
   n is 40-500;
   each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
      each R^{b} is independently —CH₃, a saturated or unsaturated alkyl moiety, an alkyne containing moiety, an azide containing moiety, a protected amine moiety, an aldehyde or protected aldehydes containing moiety, a thiol or protected thiol containing moiety, a cyclooctyne containing moiety, difluorocylcooctyne containing moiety, a nitrile oxide containing moiety, an oxanorbornadiene containing moiety, or an alcohol or protected alcohol containing moiety.

In certain embodiments, the y group is about 1 to about 200. In certain embodiments, the y group is about 25. In certain embodiments, the y group is about 10. In certain embodiments, the y group is about 20. In certain embodiments, the y group is about 15. In other embodiments y is about 1 to about 25. In other embodiments, y is about 50. According to yet another embodiment, y is about 25-75. In other embodiments, y is about 100. In other embodiments, y is selected from 10±5, 15±5, 25±5, 50±5, 75±10, 100±10, or 125±10.

As defined generally above, the n group is 40-500. In certain embodiments n is about 225. In some embodiments, n is about 275. In other embodiments, n is about 110. In other embodiments, n is about 40 to about 60. In other embodiments, n is about 60 to about 90. In still other embodiments, n is about 90 to about 150. In other embodiments, n is about 150 to about 200. In some embodiments, n is about 200 to about 300, about 300 to about 400, about 400 to about 500. In still other embodiments, n is about 250 to about 280. In other embodiments, n is about 300 to about 375. In other embodiments, n is about 400 to about 500. In certain embodiments, n is selected from 50±10. In other embodiments, n is selected from 80±10, 115±10, 180±10, 225±10, 275±10, or 450±10.

In certain embodiments, the R^{b} group is —CH₂CH₂N₃. In other embodiments, the R^{b} group is —CH₃. In yet other embodiments, the R^{b} group is mixture of both —N₃ and —CH₃.

In certain embodiments, the G group is a valence bond. In other embodiments, the G group of is a carbonyl group. In other embodiments, the G group is represented by a moiety in Table 4.

TABLE 4

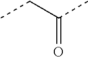

o

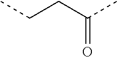

p

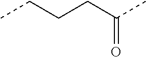

q

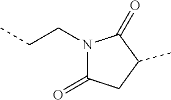

r

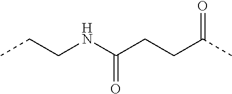

s

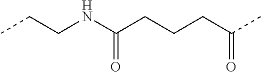

t

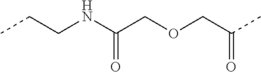

u

In some embodiments, the present invention provides a cationic polymer of formula IV, or a salt thereof, wherein each variable is as defined and described herein, both singly and in combination.

In some embodiments, the present invention provides a polyplex having a polynucleotide encapsulated therein, comprising a cationic polymer of formula IV, or a salt thereof, wherein each variable is as defined and described herein, both singly and in combination, and PEI.

In some embodiments, the present invention provides a polyplex having a polynucleotide encapsulated therein, comprising a cationic polymer of formula IV, or a salt thereof, wherein each variable is as defined and described herein, both singly and in combination, and linear PEI.

In some embodiments, the present invention provides a polyplex having a polynucleotide encapsulated therein, comprising a cationic polymer of formula IV, or a salt thereof, wherein each variable is as defined and described herein, both singly and in combination, and branched PEI.

In some embodiments, the present invention provides a polyplex having a polynucleotide encapsulated therein, comprising a cationic polymer of formula II and formula IV, or a salt thereof, wherein each variable is as defined and described herein, both singly and in combination.

In some embodiments, the present invention provides a polyplex having a polynucleotide encapsulated therein, comprising a cationic polymer of formula III and formula IV, or a salt thereof, wherein each variable is as defined and described herein, both singly and in combination.

In some embodiments, the present invention provides a polyplex having a polynucleotide encapsulated therein, comprising a cationic polymer of formula IV, or a salt thereof, wherein each variable is as defined and described herein, both singly and in combination, and arginine.

In some embodiments, the present invention provides a polyplex having a polynucleotide encapsulated therein, comprising a cationic polymer of formula IV, or a salt thereof, wherein each variable is as defined and described herein, both singly and in combination, and lysine.

In some embodiments, the present invention provides a polyplex having a polynucleotide encapsulated therein, comprising a cationic polymer of formula IV, or a salt thereof, wherein each variable is as defined and described herein, both singly and in combination, and orninthine.

Exemplary polymers, or salts thereof, of Formula IV are set forth in Table 5, wherein m is 10-250 and y is 10-250.

TABLE 5

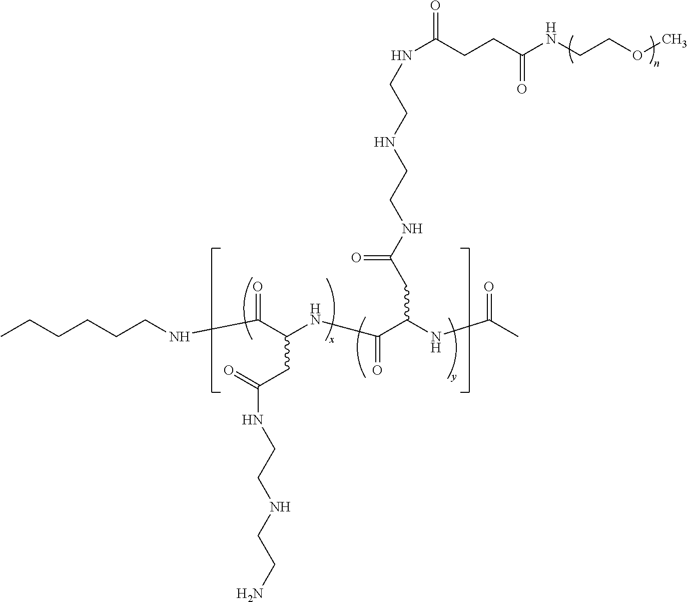

v

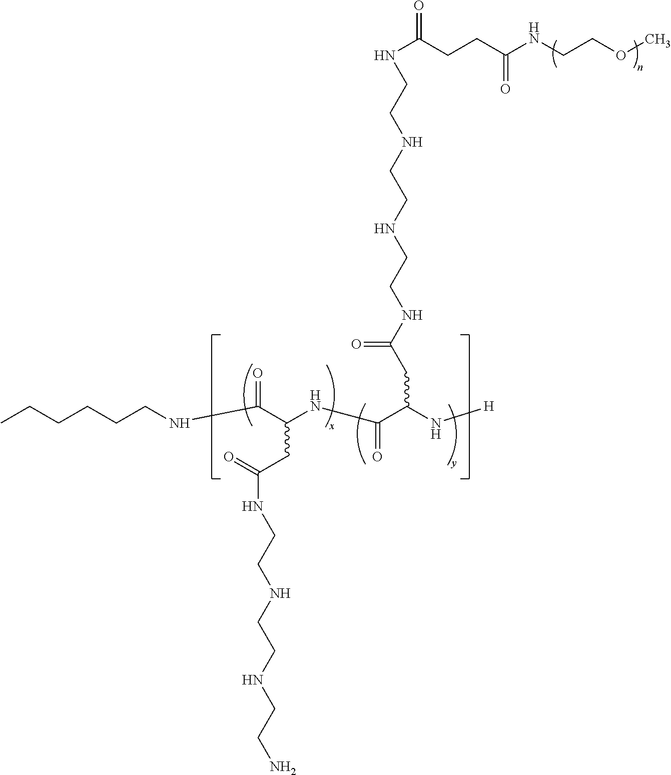

w

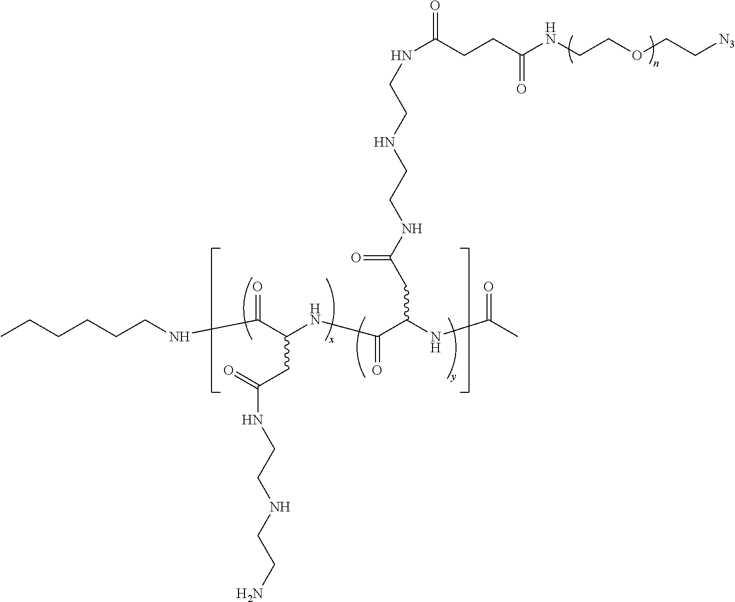

x

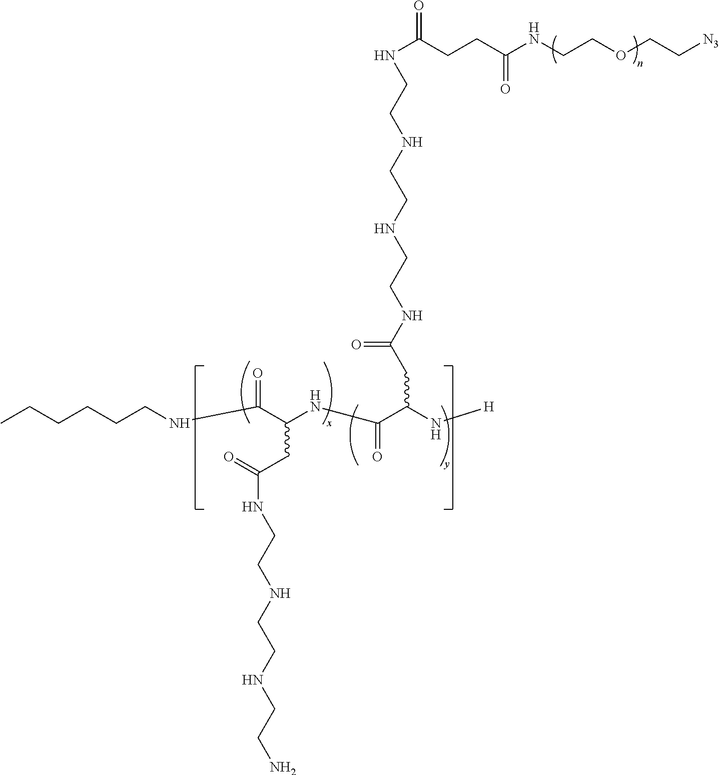

y

In certain embodiments, the present invention provides method of preparation for a PEG-conjugated polyplex having a polynucleotide encapsulated therein, comprising a cationic polymer of formula IV or a salt thereof:

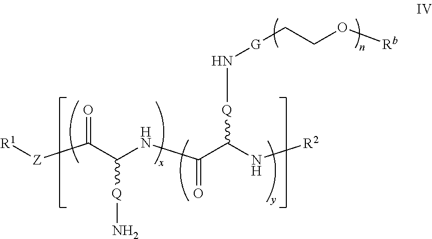

wherein each of R¹, Q, Z, G, m, y, n, R^{b} and R² is as defined above and as described in classes and subclasses herein, both singly and in combination;

comprising the steps of:
(1) providing a polyplex having a polynucleotide encapsulated therein, comprising a cationic polymer of formula I, as defined above and described in classes and subclasses herein;
(2) optionally adjusting the pH of the polyplex solution to pH 4.0-9.0; and
(3) conjugating a compound of formula V to the polyplex by reaction of the electrophile of formula V and an amine group of Formula I to afford the cationic polymer of formula IV,

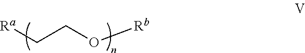

wherein each of R^{b} and n is as defined above and as described in classes and subclasses herein, both singly and in combination;
   R^{a} is a suitable electrophile; and

As generally described above, an electrophile of R^{a} is generally described as a moiety capable of reacting with a nucleophile to form a new covalent bond. In certain embodiments, a suitable electrophile is one that is capable of reacting with an amine derivative. Suitable electrophiles include, but are not limited to maleimide derivatives, activated ester moieties, esters, and aldehyde moieties.

It will be appreciated by one skilled in the art that the copolymer of formula IV represents a random, mixed copolymer of free amines or ammonium salts and amines that have reacted with a compound of formula V to provide a covalent bond attaching the grafted PEG chain to the poly(amino acid) backbone. Thus, a mixture of free amines or ammonium salts and PEG chains now represents the side chains of the poly(amino acid) copolymer. It will be appreciated that if and only if the m group of formula IV is zero, then each and every amine would have reacted with a compound of formula V and no free amine or ammoniums salts would exist in formula IV.

Exemplary compounds of formula V can be found in Table 6a and 6b, wherein each n is independently 40-500.

TABLE 6a

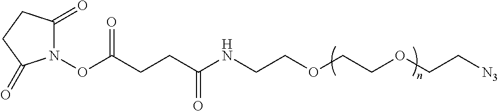

i

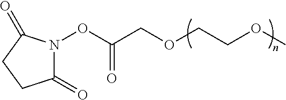

ii

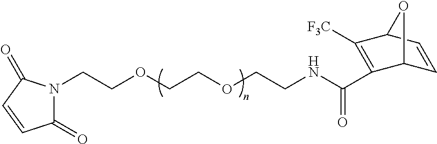

iii

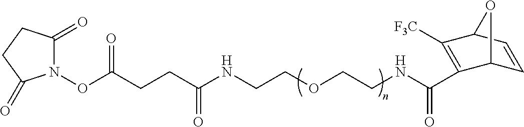

vi

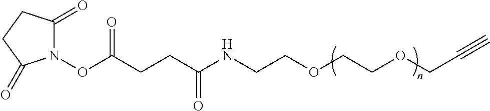

v

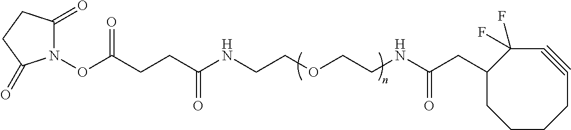

iv

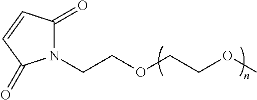

vii

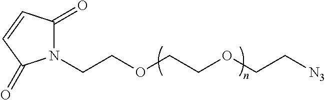

viii

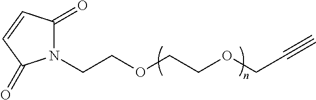

ix

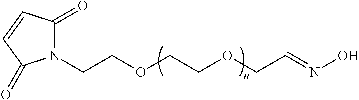

x

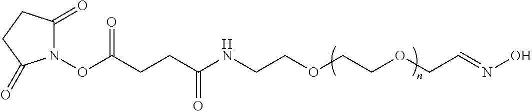

xi

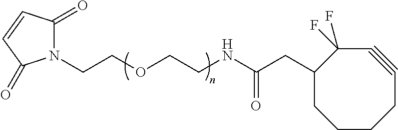

xii

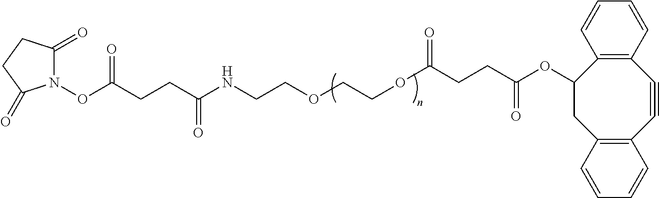

xiii

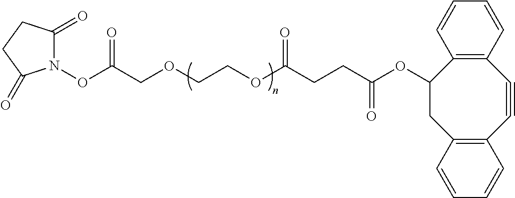

xiv

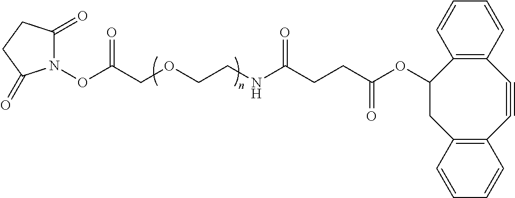

xv

TABLE 6b

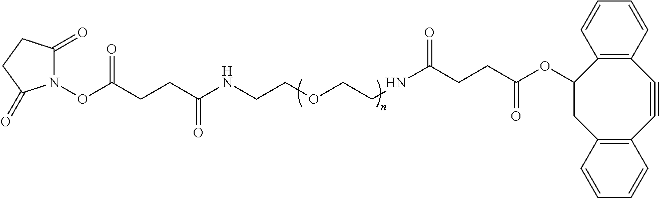

xvi

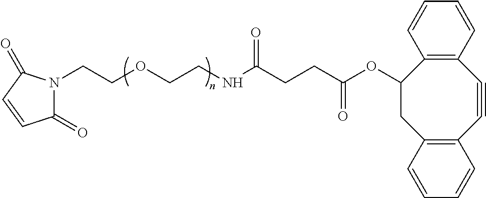

xvii

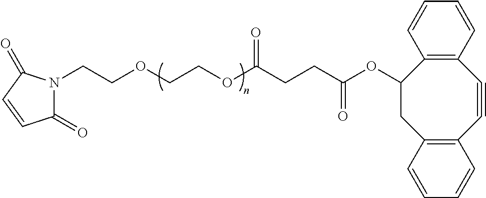

xviii

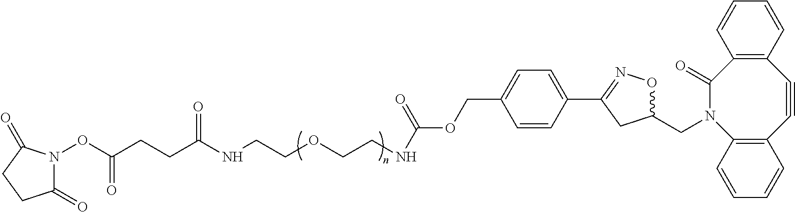

xix

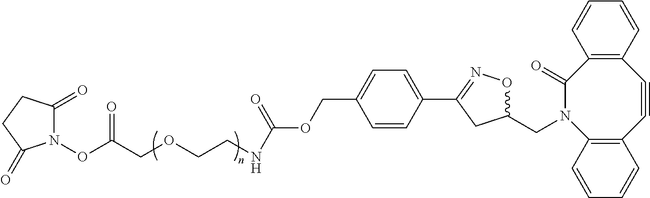

xx

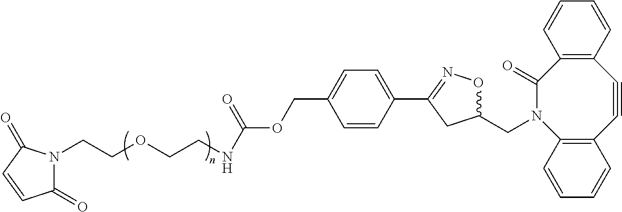

xxi

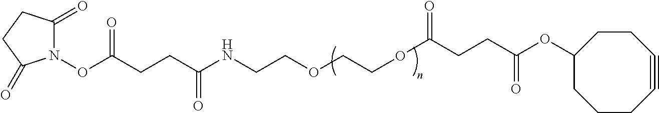

xxii

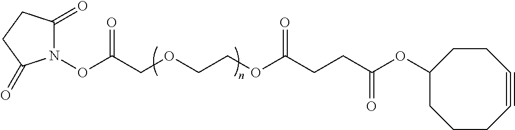

xxiii

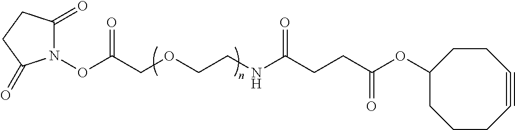

xxiv

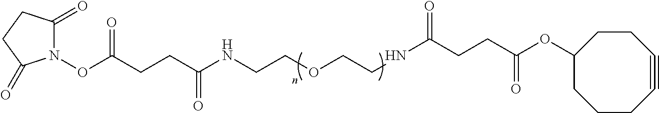

xxv

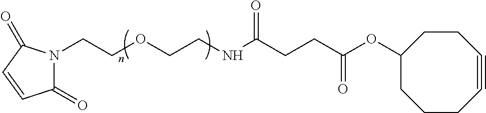

xxvi

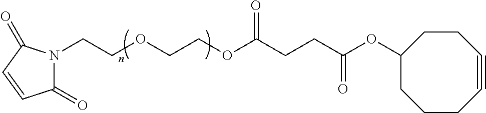

xxvii

D. Targeting Group Attachment

PEG-conjugated polyplexes described herein can be modified to enable active cell-targeting to maximize the benefits of current and future therapeutic agents. Because these polyplexes typically possess diameters greater than 20 nm, they exhibit dramatically increased circulation time when compared to stand-alone drugs due to minimized renal clearance. This unique feature of nanovectors leads to selective accumulation in diseased tissue, especially cancerous tissue due to the enhanced permeation and retention effect (“EPR”). The EPR effect is a consequence of the disorganized nature of the tumor vasculature, which results in increased permeability of polymer therapeutics and drug retention at the tumor site. In addition to passive cell targeting by the EPR effect, these polyplexes are designed to actively target tumor cells through the chemical attachment of targeting groups to the polyplex periphery. The incorporation of such groups is most often accomplished through end-group functionalization of the PEG block using chemical conjugation techniques. Like viral particles, polyplexes functionalized with targeting groups utilize receptor-ligand interactions to control the spatial distribution of the polyplexses after administration, further enhancing cell-specific delivery of therapeutics. In cancer therapy, targeting groups are designed to interact with receptors that are over-expressed in cancerous tissue relative to normal tissue such as folic acid, oligopeptides, sugars, and monoclonal antibodies. See Pan, D.; Turner, J. L.; Wooley, K. L. Chem. Commun. 2003, 2400-2401; Gabizon, A.; Shmeeda, H.; Horowitz, A. T.; Zalipsky, S. Adv. Drug Deliv. Rev. 2004, 56, 1177-1202; Reynolds, P. N.; Dmitriev, I.; Curiel, D. T. Vector. Gene Ther. 1999, 6, 1336-1339; Derycke, A. S. L.; Kamuhabwa, A.; Gijsens, A.; Roskams, T.; De Vos, D.; Kasran, A.; Huwyler, J.; Missiaen, L.; de Witte, P. A. M. T J. Nat. Cancer Inst. 2004, 96, 1620-30; Nasongkla, N., Shuai, M., Ai, H.; Weinberg, B. D. P., J.; Boothman, D. A.; Gao, J. Angew. Chem. Int. Ed. 2004, 43, 6323-6327; Jule, E.; Nagasaki, Y.; Kataoka, K. Bioconj. Chem. 2003, 14, 177-186; Stubenrauch, K.; Gleiter, S.; Brinkmann, U.; Rudolph, R.; Lilie, H. Biochem. J. 2001, 356, 867-873; Kurschus, F. C.; Kleinschmidt, M.; Fellows, E.; Dornmair, K.; Rudolph, R.; Lilie, H.; Jenne, D. E. FEBS Lett. 2004, 562, 87-92; and Jones, S. D.; Marasco, W. A. Adv. Drug Del. Rev. 1998, 31, 153-170.

The R^{b} moiety can be used to attach targeting groups for cell specific delivery including, but not limited to, proteins, oliogopeptides, antibodies, monosaccarides, oligosaccharides, vitamins, or other small biomolecules. Such targeting groups include, but are not limited to monoclonal and polyclonal antibodies (e.g. IgG, IgA, IgM, IgD, IgE antibodies), sugars (e.g. mannose, mannose-6-phosphate, galactose), proteins (e.g. Transferrin), oligopeptides (e.g. cyclic and acylic RGD-containing oligopeptides), and vitamins (e.g. folate).

In other embodiments, the R^{b} moiety is conjugated to biomolecules which promote cell entry and/or endosomal escape. Such biomolecules include, but are not limited to, oligopeptides containing protein transduction domains such as the HIV Tat peptide sequence (GRKKRRQRRR) or oligoarginine (RRRRRRRRR). Oligopeptides which undergo conformational changes in varying pH environments such oligohistidine (HHHHH) also promote cell entry and endosomal escape.

Compounds having R^{b} moieties suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the R^{b} groups to a macromolecule via Click chemistry or metal free click chemistry.

According to one embodiment, the R^{b} moiety is an azide-containing group. According to another embodiment, the R^{b} moiety is an alkyne-containing group. In certain embodiments, the R^{b} moiety has a terminal alkyne moiety. In other embodiments, the R^{b} moiety is an alkyne moiety having an electron withdrawing group. Accordingly, in such embodiments, the R^{b} moiety is

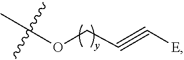

wherein E is an electron withdrawing group and y is 0-6. Such electron withdrawing groups are known to one of ordinary skill in the art. In certain embodiments, E is an ester. In other embodiments, the R^{b} moiety is

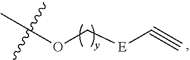

wherein E is an electron withdrawing group, such as a —C(O)O— group and y is 0-6.

In other embodiments, the R^{b} moiety is suitable for metal free click chemistry (also known as copper free click chemistry). Examples of such chemistries include cyclooctyne derivatives (Codelli, et. al. J. Am. Chem. Soc., 2008, 130, 11486-11493; Jewett, et. al. J. Am. Chem. Soc., 2010, 132, 3688-3690; Ning, et. al. Angew. Chem. Int. Ed., 2008, 47, 2253-2255), difluoro-omanorbornene derivatives (van Berkel, et. al. ChemBioChem, 2007, 8, 1504-1508), or nitrile oxide derivatives (Lutz, et. al. Macromolecules, 2009, 42, 5411-5413). Without wishing to be bound by any particular theory, it is believed that the use of metal free click conditions offers certain advantages for the encapsulation of polynucleotides. Such functionalized PEG derivatives suitable for metal free click chemistry are described in detail in United States Patent Publication No. US 2011-0224383, filed Mar. 11, 2011, the entirety of which is hereby incorporated herein by reference.

Certain metal-free click moieties are known in the literature. Examples include 4-dibenzocyclooctynol (DIBO)

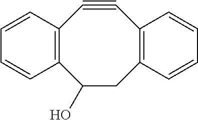

(from Ning et. al; Angew Chem Int Ed, 2008, 47, 2253); difluorinated cyclooctynes (DIFO or DFO)

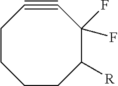

(from Codelli, et. al.; J. Am. Chem. Soc. 2008, 130, 11486-11493); biarylazacyclooctynone (BARAC)

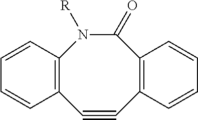

(from Jewett et. al.; J. Am. Chem. Soc. 2010, 132, 3688); or bicyclononyne (BCN)

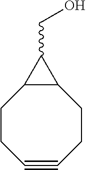

### (From Dommerholt, et. al.; Angew Chem Int Ed, 2010, 49, 9422-9425).

In certain embodiments, the present invention provides a targeted PEG-conjugated cationic polymer of formula VI or a salt thereof:

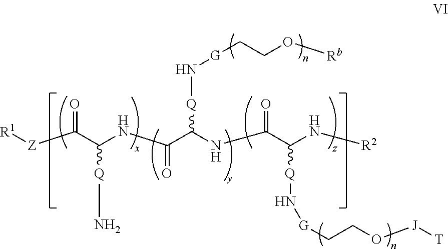

wherein each of R¹, Q, Z, G, m, y, n, R^{b} and R² is as defined above and as described in classes and subclasses herein, both singly and in combination;
z is 1-200;
each J is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
T is a targeting group.

In certain embodiments, the z group is about 1 to about 200. In certain embodiments, the z group is about 25. In certain embodiments, the z group is about 10. In certain embodiments, the z group is about 20. In certain embodiments, the z group is about 15. In other embodiments z is about 1 to about 25. In other embodiments, z is about 50. According to yet another embodiment, z is about 25-75. In other embodiments, z is about 100. In other embodiments, z is selected from 10±5, 15±5, 25±5, 50±5, 75±10, 100±10, or 125±10.

In certain embodiments, the J group is a valence bond as described above. In certain embodiments, the J group is a methylene group. In other embodiments, the J group is a carbonyl group. In certain embodiments, the J group is a valence bond. In other embodiments, the J group is represented by a moiety in Table 7.

TABLE 7

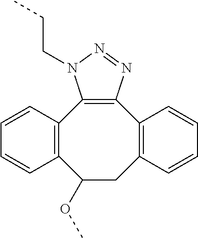

aa

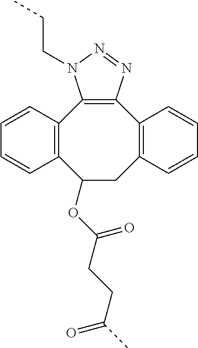

bb

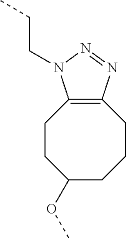

cc

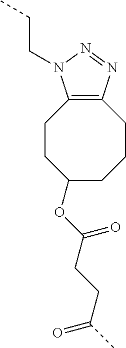

dd

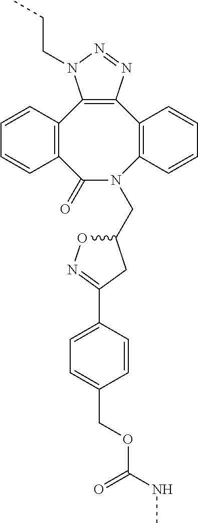

ee

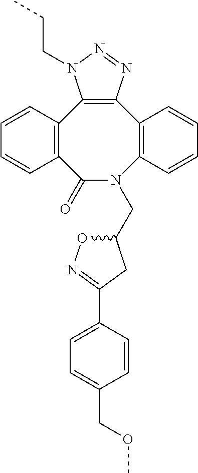

ff

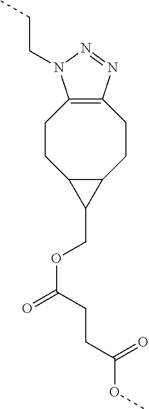

gg

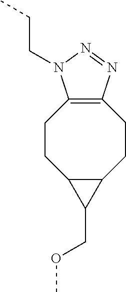

hh

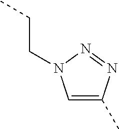

ii

In some embodiments, the present invention provides a PEG-conjugated polyplex, having a polynucleotide encapsulated therein, comprised of cationic polymers of formula II and formula VI, or a salt thereof, wherein each variable is as defined and described herein, both singly and in combination.

In some embodiments, the present invention provides a PEG-conjugated polyplex, having a polynucleotide encapsulated therein, comprised of cationic polymers of formula III and formula VI, or a salt thereof, wherein each variable is as defined and described herein, both singly and in combination.

In some embodiments, the present invention provides a PEG-conjugated polyplex, having a polynucleotide encapsulated therein, comprised of cationic polymers of arginine and formula VI, or a salt thereof, wherein each variable is as defined and described herein, both singly and in combination.

In some embodiments, the present invention provides a PEG-conjugated polyplex, having a polynucleotide encapsulated therein, comprised of cationic polymers of PEI and formula VI, or a salt thereof, wherein each variable is as defined and described herein, both singly and in combination.

In some embodiments, the present invention provides a PEG-conjugated polyplex, having a polynucleotide encapsulated therein, comprised of cationic polymers of lysine and formula VI, or a salt thereof, wherein each variable is as defined and described herein, both singly and in combination.

In some embodiments, the present invention provides a PEG-conjugated polyplex, having a polynucleotide encapsulated therein, comprised of cationic polymers of ornithine and formula VI, or a salt thereof, wherein each variable is as defined and described herein, both singly and in combination.

It will be appreciated by one skilled in the art the copolymer of formula VI is a mixed, random copolymer comprised of side chain groups containing free amines or ammonium salts; conjugated PEG chains; and conjugated PEG chains with a terminal targeting group moiety. Furthermore, it is understood that m of formula VI represents the number of free amines or ammonium salts; that y of formula VI represents the number of repeats having pendant PEG chains; and that z of formula VI represents the number of repeats that have a pendant PEG chain possessing a terminal targeting group.

### 4. Uses, Methods, and Compositions

As described herein, polyplexes of the present invention can encapsulate a wide variety of therapeutic agents useful for treating a wide variety of diseases. In certain embodiments, the present invention provides a nucleotide-loaded polyplex, as described herein, wherein said polyplex is useful for treating the disorder for which the nucleotide is known to treat. According to one embodiment, the present invention provides a method for treating one or more disorders selected from pain, inflammation, arrhythmia, arthritis (rheumatoid or osteoarthritis), atherosclerosis, restenosis, bacterial infection, viral infection, depression, diabetes, epilepsy, fungal infection, gout, hypertension, malaria, migraine, cancer or other proliferative disorder, erectile dysfunction, a thyroid disorder, neurological disorders and hormone-related diseases, Parkinson's disease, Huntington's disease, Alzheimer's disease, a gastro-intestinal disorder, allergy, an autoimmune disorder, such as asthma or psoriasis, osteoporosis, obesity and comorbidities, a cognitive disorder, stroke, AIDS-associated dementia, amyotrophic lateral sclerosis (ALS, Lou Gehrig's disease), multiple sclerosis (MS), schizophrenia, anmiety, bipolar disorder, tauopothy, a spinal cord or peripheral nerve injury, myocardial infarction, cardioxyocyte hypertrophy, glaucoma, an attention deficit disorder (ADD or ADHD), a sleep disorder, reperfusion/ischemia, an angiogenic disorder, or urinary incontinence, comprising administering to a patient a PEG-conjugated polyplex, wherein said polyplex encapsulates a therapeutic agent suitable for treating said disorder.

In certain embodiments, the present invention provides a method for treating one or more disorders selected from an autoimmune disease, an inflammatory disease, a metabolic disorder, a psychiatric disorder, diabetes, an angiogenic disorder, tauopothy, a neurological or neurodegenerative disorder, a spinal cord injury, glaucoma, baldness, or a cardiovascular disease, comprising administering to a patient an optionally targeted, PEG-covered polyplex wherein said polyplex encapsulates a therapeutic polynucleotide suitable for treating said disorder.

In certain embodiments, nucleotide-loaded polyplexes of the present invention are useful for treating cancer. Accordingly, another aspect of the present invention provides a method for treating cancer in a patient comprising administering to a patient an optionally targeted, PEG-covered polyplex wherein said polyplex encapsulates a therapeutic polynucleotide suitable for treating said cancer. In certain embodiments, the present invention relates to a method of treating a cancer selected from breast, ovary, cervim, prostate, testis, genitourinary tract, esophagus, larynm, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynm (oral), lip, tongue, mouth, pharynm, small intestine, colon-rectum, large intestine, rectum, brain and central nervous system, and leukemia, comprising administering a polyplex in accordance with the present invention wherein said polyplex encapsulates a therapeutic polynucleotide suitable for treating said cancer.

Compositions

In certain embodiments, the invention provides a composition comprising a polyplex of this invention or a pharmaceutically acceptable derivative thereof and a pharmaceutically acceptable carrier, adjuvant, or vehicle. In certain embodiments, a composition of this invention is formulated for administration to a patient in need of such composition. In certain embodiments, a composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hemanoate, hydrochloride, hydrobromide, hydroiodide, 2-hydroxyethanesulfonate, lactate, maleate, malonate, methanesulfonate, 2-naphthalenesulfonate, nicotinate, nitrate, omalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as omalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for Example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In certain embodiments, pharmaceutically acceptable compositions of the present invention are enterically coated.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the drug can be administered to a patient receiving these compositions.

It will be appreciated that dosages typically employed for the encapsulated drug are contemplated by the present invention. In certain embodiments, a patient is administered a drug-loaded polyplex of the present invention wherein the dosage of the drug is equivalent to what is typically administered for that drug. In other embodiments, a patient is administered a drug-loaded polyplex of the present invention wherein the dosage of the drug is lower than is typically administered for that drug.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In order that the invention described herein may be more fully understood, the following examples are set forth. It will be understood that these examples are for illustrative purposes only and are not to be construed as limiting this invention in any manner.

### EXAMPLES

### Example 1

### Preparation of Bifunctional PEGs of the Present Invention

As described generally above, multiblock copolymers of the present invention are prepared using the heterobifunctional PEGs described herein and in U.S. Pat. No. 7,612,153, filed Oct. 24, 2005, published as WO2006/047419 on May 4, 2006 and published as US 20060142506 on Jun. 29, 2006, the entirety of which is hereby incorporated herein by reference. Additional heterobifunctional PEGs are described in United States patent publication US 2011-0224383, filed Mar. 11, 2011, and in U.S. patent application Ser. No. 61/584,412, filed Jan. 9, 2012, the entirety of both applications are hereby incorporated by reference. The preparation of multiblock polymers in accordance with the present invention is accomplished by methods known in the art, including those described in detail in U.S. patent application Ser. No. 11/325,020, filed Jan. 4, 2006, published as WO2006/74202 on Jul. 13, 2006 and published as US 20060172914 on Aug. 3, 2006, the entirety of which is hereby incorporated herein by reference.

### Example 2

### Preparation of Poly(Asp-DET) of the Present Invention

As described generally above, poly(Asp-DET) polymers of the present invention are prepared in U.S. patent application Ser. No. 13/047,733, filed Mar. 14, 2011, the entirety of which is hereby incorporated herein by reference. The preparation of multiblock polymers in accordance with the present invention is accomplished by methods known in the art, including those described in detail in United States patent publication number US 2011-0229528, filed Mar. 14, 2011, the entirety of which is hereby incorporated herein by reference.

### Example 3

### Formulation of Polymer/Nucleic Acid Polyplexes

Poly(D/L Asp-DET)/DNA polyplexes were prepared by adding equal volumes of Poly(D/L Asp-DET) solution (dissolved in dH₂O) and plasmid DNA solution (200 μg/mL in dH₂O) at the appropriate N:P ratio. Polymer was added to DNA solution, for a final volume of 200 μL, and incubated at room temperature for at least 30 min to allow polyplex formation. PEG-polyplexes were formed by incubating 200 μL of Poly(D/L Asp-DET)/DNA N:P 10 polyplexes with 50 μL of Azide-12k PEG-NHS (60 mg/mL in dH₂O, refs) for 3 hr with shaking at room temperature. Un-reacted PEG was removed by ultrafiltration using a Vivaspin 500 100,000 MWCO filters (Sartorius Stedim Biotech GmbH, Germany), and PEG-polyplexes were diluted with dH₂O to a final volume of 200 μL to achieve equal volumes for all samples.

### Example 4

### Gel Retardation Experiments and Ethidium Bromide Exclusion Assays

Polyplexes containing Luciferase plasmid DNA (pGL4; Promega, Madison, Wis.) were prepared (as described in Example 2) at various N:P ratios. Five μL of each formulation was run on a 1% agarose gel and visualized by ethidium bromide staining. For ethidium bromide exclusion assays, DNA only (100 μg/mL in H₂O) and polyplex solutions were diluted 1:4 with dH₂O to a final volume of 50 μL. Fifty μL of ethidium bromide (2 ug/mL in H2O), was added to the solutions, mixed and incubated at room temperature for 20 min. The fluorescence intensity of triplicate samples was measured at λ=580 nm (excitation at λ=540) with a spectrofluorometer (FLUORstar OPTIMA, BMG Labtech Inc.). Relative Fluorescence Units (RFU) were calculated using: RFU=(Flₛₐₘₚₗₑ−Fl₀)/(Fl_{DNA}−Fl₀), where Flₛₐₘₚₗₑ, Fl₀ and Fl_{DNA} represent the fluorescence intensity of the samples, background and free plasmid DNA, respectively.

### Example 5

### Dynamic Light Scattering (DLS) and Zeta-Potential Measurements

Polyplex sizes were measured using a DynaPro Dynamic Light Scattering Plate Reader (Wyatt Technology Corporation, Santa Barbara, Calif.), and determined every hr for eight hr with ten 30 sec acquisitions at 37° C. Zeta-potential measurements were determined using a Zetasizer Nano instrument (Malvern Instruments Ltd, UK), and represent the average of three runs at 25° C.

### Example 6

### Salt Addition and Centrifugation Studies

Non- and PEG-polyplex samples (as described above in Example 3), along with complexes made with JetPEI and Superfect, were spiked with 5 M NaCl for a final 150 mM concentration. Experiments using JetPEI (Polyplus-transfection Inc. New York, N.Y.) and Superfect (Qiagen, Valencia, Calif.) were also performed using the manufacturers' recommended protocols. Samples were incubated, initial UV absorbance at 260 nm measured, and samples centrifuged at intervals of increasing g forces for 1 minute. After each centrifugation step, supernatant UV absorbance was determined at 260 nm. A/Ao ratios were calculated for each centrifugation step. Ao; initial sample absorbance value at 260 nm, A; absorbance of sample supernatant after each centrifugation. After the final centrifugation, supernatant samples were resolved on a 1% agarose/ethidium bromide gel. Heparin was added to duplicate samples to dissociate DNA from polymers. Poly; Poly(d/l Asp-DET)/DNA polyplex, DNA M; 1 kb DNA ladder.

### Example 7

### Comparison of DNA Complexation Using Either PEI or Poly(D/L Asp-DET) Polymers

PEG-Polyplexes using Poly(D/L Asp-DET) polymer were formulated as described in Example 3. PEG-Polyplexes using 22 kDa linear or 25 kDa branched PEIs were prepared by adding equal volumes of PEI solution (dissolved in dH₂O) and plasmid DNA solution (200 μg/mL in dH₂O) at the appropriate N:P ratio. PEI polymer was added to DNA solution, for a final volume of 200 μL, and incubated at room temperature for at least 30 min to allow polyplex formation. PEG-PEI Polyplexes were prepared as described in Example 3. Samples (0.5 μg of DNA) were electrophoresed in a 1% agarose gel as described for the gel retardation experiment (Example 5), FIG. 1A. Unlike PEIs, the covalent attachment of PEG did not affect binding affinity of Poly(D/L Asp-DET) polymer to DNA FIG. 1A. Polyplex samples were centrifuged following salt addition and incubation as described in Example 6. After centrifugation, heparin was added to supernatant to dissociate DNA from polymers and the samples were resolved on a 1% agarose/ethidium bromide gel, shown in FIG. 1B. Only PEG-Poly(D/L Asp-DET)/DNA Polyplex samples remained in solution and contained intact DNA following the addition of salt. Poly(D/L Asp-DET) polymers allow for necessary PEG coverage to avoid salt induced aggregation.

### Example 8

### Comparison of Polyplex and PEG-Polyplex Size and Morphology

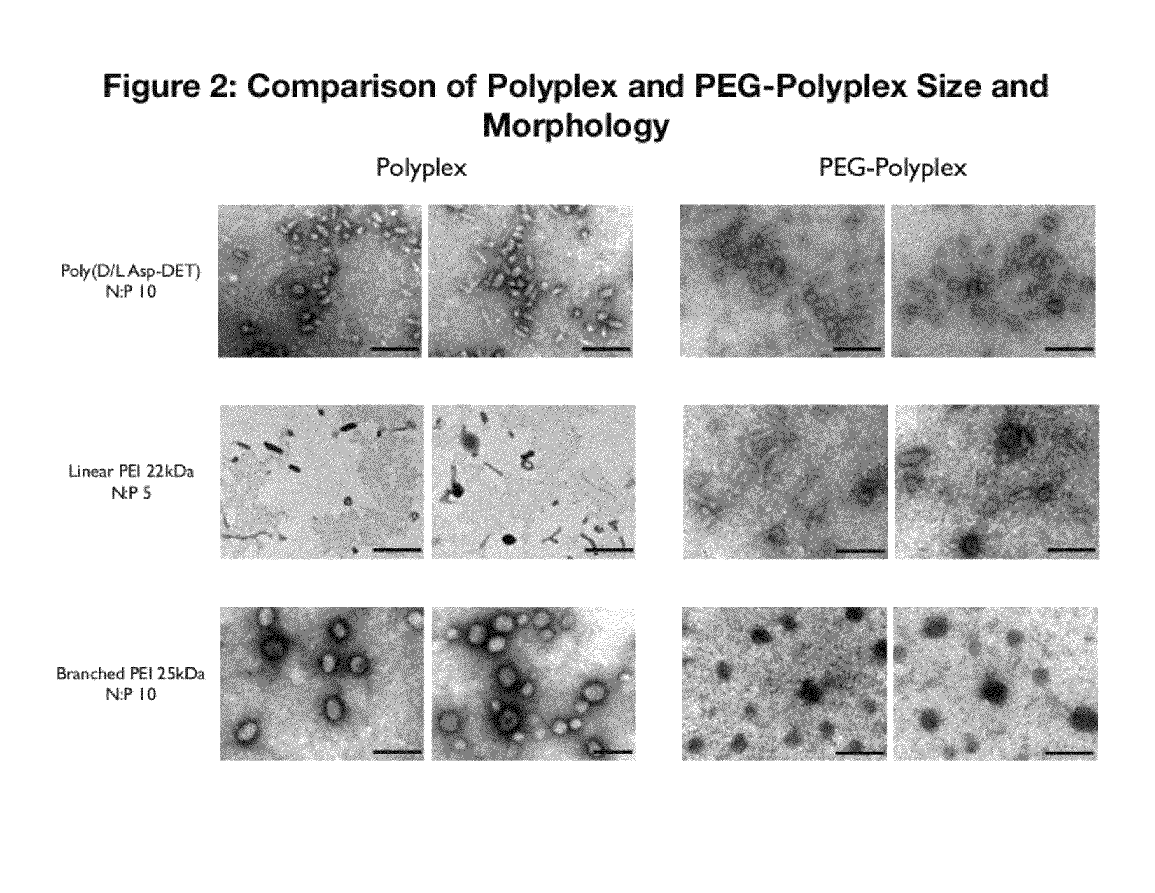

TEM analysis of Poly(D/L Asp-DET), 22 kDa linear PEI or 25 kDa branched PEI Polyplexes and PEG-Polyplexes, FIG. 2. PEG-Polyplexes created with Poly(D/L Asp-DET) polymers formed the smallest and most uniformed morphologies. Bar=200 nm.

### Example 9

### Co-Complexation of DNA Using Linear PEI and Poly(D/L Asp-DET) Polymers

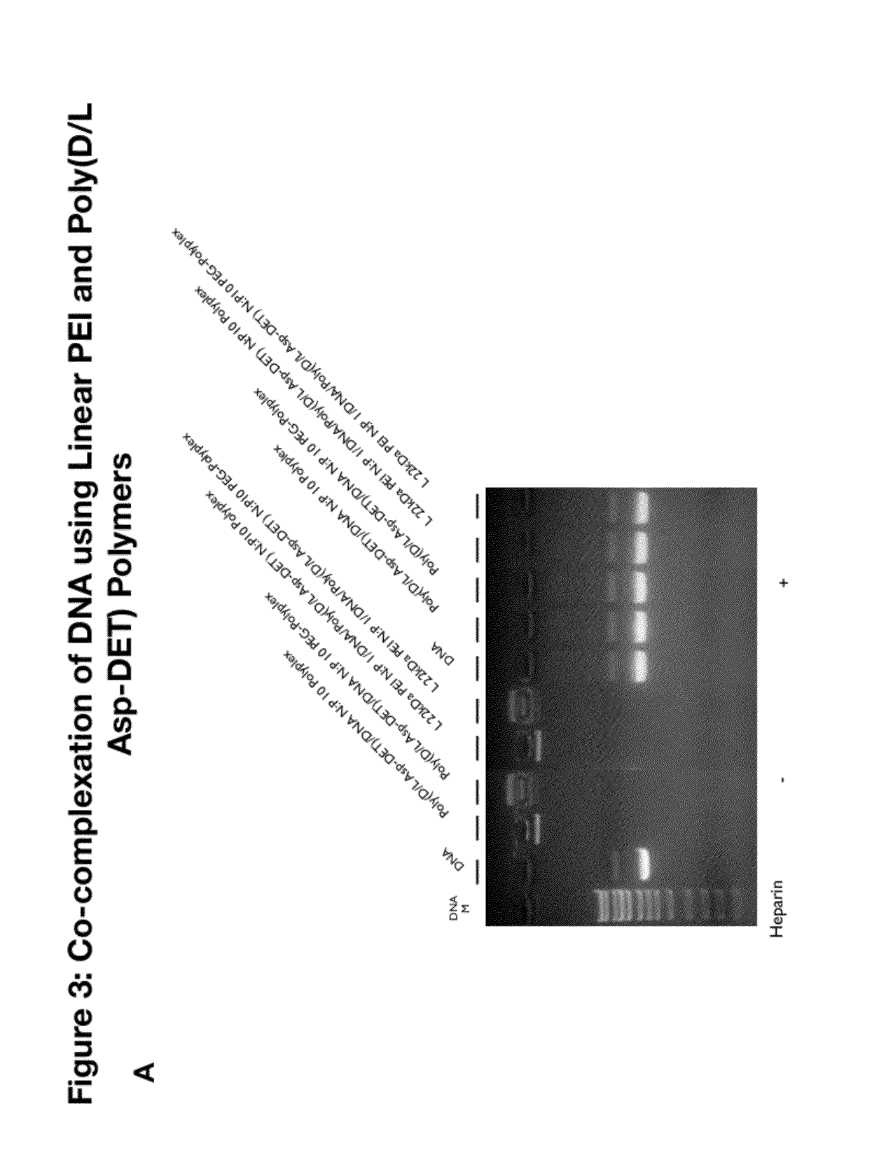

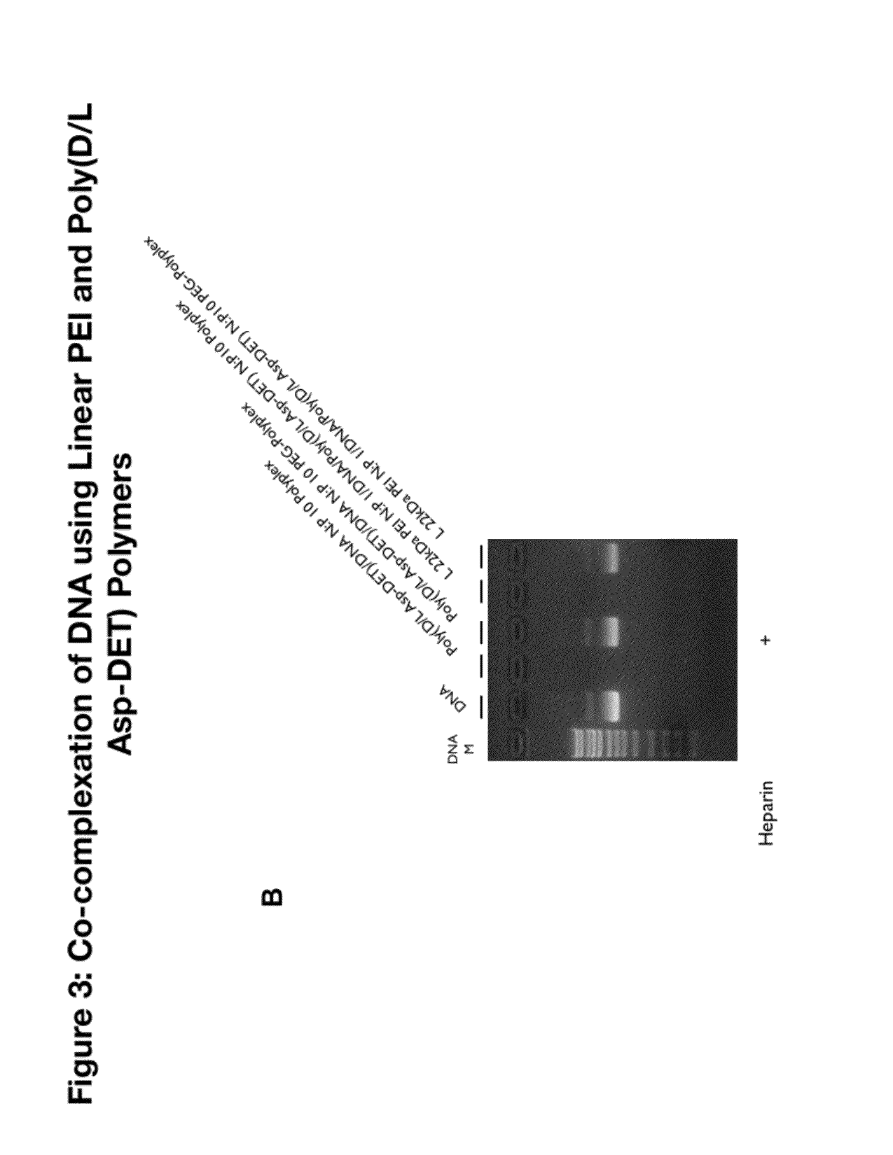

PEG-Polyplexes using Poly(D/L Asp-DET) polymer were formulated as described in Example 3. PEG-Polyplexes using both 22 kDa linear PEI and Poly(D/L Asp-DET) were prepared by adding PEI solution (dissolved in dH₂O) to plasmid DNA solution (200 μg/mL in dH₂O) at N:P ratio 1 and incubating at room temperature for at least 30 min. Poly(D/L Asp-DET) polymer solution (dissolved in dH₂O) was then added and incubated for an additional 30 min at room temperature to complete polyplex formation. PEG-PEI/Poly(D/L Asp-DET) Polyplexes were prepared as described in Example 3. Samples (0.5 μg of DNA) were electrophoresed in a 1% agarose gel as described for the gel retardation experiment (Example 4), FIG. 3A. The covalent attachment of PEG was not affected in co-complexed samples. Polyplex samples were centrifuged following salt addition and incubation as described in Example 6. After centrifugation, heparin was added to supernatant to dissociate DNA from polymers and the samples were resolved on a 1% agarose/ethidium bromide gel, shown in FIG. 3B. PEG-PEI/Poly(D/L Asp-DET)/DNA Polyplex samples remained in solution and contained intact DNA following the addition of salt.

We claim:

1. A polyplex comprised of two or more cationic polymers, having a polynucleotide encapsulated therein.

2. A polyplex comprised of two or more cationic polymers, having a polynucleotide encapsulated therein, wherein the cationic polymers comprise poly(ethylene imine) (PEI) and a polymer of Formula I: 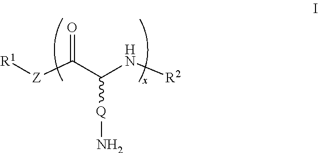
wherein:
m is 10-250;
each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
R¹ is hydrogen, —N₃, —CN, a suitable amine protecting group, a protected aldehyde, a protected hydroxyl, a suitable hydroxyl protecting group, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide.

3. The polyplex of claim 2, wherein PEI is linear PEI.

4. The polyplex of claim 2, wherein PEI is branched PEI.

5. The polyplex of claim 2, wherein the cationic polymers consist of poly(ethylene imine) (PEI) and a cationic polymer of Formula I.

6. A polyplex comprised of two or more cationic polymers, having a polynucleotide encapsulated therein, wherein the cationic polymers comprise PEI and a polymer of Formula IV: 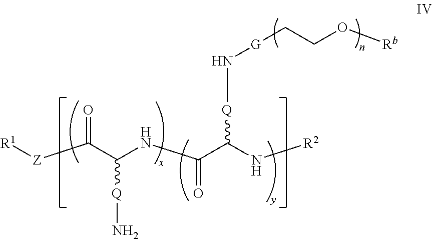
wherein:
y is 1-200;
n is 40-500;
m is 10-250;
each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
R¹ is hydrogen, —N₃, —CN, a suitable amine protecting group, a protected aldehyde, a protected hydroxyl, a suitable hydroxyl protecting group, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide.
each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each R^{b} is independently —CH₃, a saturated or unsaturated alkyl moiety, an alkyne containing moiety, an azide containing moiety, a protected amine moiety, an aldehyde or protected aldehydes containing moiety, a thiol or protected thiol containing moiety, a cyclooctyne containing moiety, difluorocylcooctyne containing moiety, a nitrile omixe containing moiety, an oxanorbornadiene containing moiety, or an alcohol or protected alcohol containing moiety.

7. The polyplex of claim 6, wherein PEI is linear PEI.

8. The polyplex of claim 6, wherein PEI is branched PEI.

9. The polyplex of claim 6, wherein the cationic polymers consist of poly(ethylene imine) (PEI) and a cationic polymer of Formula IV.
